SEQUENCE LISTING (42 sequences)

SEQ ID NO: 1 - DNA, 726 bp, Homo sapiens
atgtccatgttgttctacactctgatcacagcttttctgatcggcatacaggcggaaccacactcagagagcaatgtccctgcaggacacaccatcccccaagcccactggactaaacttcagcattcccttgacactgcccttcgcagagcccgcagcgccccggcagcggcgatagctgcacgcgtggcggggcagacccgcaacattactgtggaccccaggctgtttaaaaagcggcgactccgttcaccccgtgtgctgtttagcacccagcctccccgtgaagctgcagacactcaggatctggacttcgaggtcggtggtgctgcccccttcaacaggactcacaggagcaagcggtcatcatcccatcccatcttccacaggggcgaattctcggtgtgtgacagtgtcagcgtgtgggttggggataagaccaccgccacagacatcaagggcaaggaggtgatggtgttgggagaggtgaacattaacaacagtgtattcaaacagtacttttttgagaccaagtgccgggacccaaatcccgttgacagcgggtgccggggcattgactcaaagcactggaactcatattgtaccacgactcacacctttgtcaaggcgctgaccatggatggcaagcaggctgcctggcggtttatccggatagatacggcctgtgtgtgtgtgctcagcaggaaggctgtgagaagagcctga

SEQ ID NO: 2 - protein, 120 aa, Homo sapiens
SSSHPIFHRGEFSVCDSVSVWVGDKTTATDIKGKEVMVLGEVNINNSVFKQYFFETKCRDPNPVDSGCRGIDSKHWNSYCTTTHTFVKALTMDGKQAAWRFIRIDTACVCVLSRKAVRRA

SEQ ID NO: 3 - protein, 120 aa, Homo sapiens
SSSHPIFHRGEESVCDSVSVWVGDKTTATDIKGKEVMVLGEVNINNSVFKQYFFETKCRDPNPVDSGCRGIDSKHWNSYCTTTHTFVKALTMDGKQAAWRFIRIDTACVCVLSRKAVRRA

SEQ ID NO: 4 - protein, 120 aa, Homo sapiens
SSSHPIFHRGEFSVCDSVSVWVGDKTTATDIGGKEVMVLGEVNINNSVFKQYFFETKCRDPNPVDSGCRGIDSKHWNSYCTTTHTFVKALTMDGKQAAWRFIRIDTACVCVLSRKAVRRA

SEQ ID NO: 5 - protein, 120 aa, Homo sapiens
SSSHPIFHRGEFSVCDSVSVWVGDKTTATDILGKEVMVLGEVNINNSVFKQYFFETKCRDPNPVDSGCRGIDSKHWNSYCTTTHTFVKALTMDGKQAAWRFIRIDTACVCVLSRKAVRRA

SEQ ID NO: 6 - protein, 120 aa, Homo sapiens
SSSHPIFHRGEFSVCDSVSVWVGDKTTATDIYGKEVMVLGEVNINNSVFKQYFFETKCRDPNPVDSGCRGIDSKHWNSYCTTTHTFVKALTMDGKQAAWRFIRIDTACVCVLSRKAVRRA

SEQ ID NO: 7 - protein, 120 aa, Homo sapiens
SSSHPIFHRGEFSVCDSVSVWVGDKTTATDIKGKEVMVLGEVNINNSVFKQYFFETKCLDPNPVDSGCRGIDSKHWNSYCTTTHTFVKALTMDGKQAAWRFIRIDTACVCVLSRKAVRRA

SEQ ID NO: 8 - protein, 120 aa, Homo sapiens
SSSHPIFHRGEFSVCDSVSVWVGDKTTATDIKGKEVMVLGEVNINNSVFKQYFFETKCADPNPVDSGCRGIDSKHWNSYCTTTHTFVKALTMDGKQAAWRFIRIDTACVCVLSRKAVRRA

SEQ ID NO: 9 - protein, 120 aa, Homo sapiens
SSSHPIFHRGEFSVCDSVSVWVGDKTTATDIKGKEVMVLGEVNINNSVFKQYFFETKCRDPNPVASGCRGIDSKHWNSYCTTTHTFVKALTMDGKQAAWRFIRIDTACVCVLSRKAVRRA

SEQ ID NO: 10 - protein, 120 aa, Homo sapiens
SSSHPIFHRGEFSVCDSVSVWVGDKTTATDIKGKEVMVLGEVNINNSVFKQYFFETKCRDPNPVGSGCRGIDSKHWNSYCTTTHTFVKALTMDGKQAAWRFIRIDTACVCVLSRKAVRRA

SEQ ID NO: 11 - protein, 120 aa, Homo sapiens
SSSHPIFHRGEFSVCDSVSVWVGDKTTATDIKGKEVMVLGEVNINNSVFKQYFFETKCRDPNPVDSGCRGIDSLHWNSYCTTTHTFVKALTMDGKQAAWRFIRIDTACVCVLSRKAVRRA

SEQ ID NO: 12 - protein, 120 aa, Homo sapiens
SSSHPIFHRGEFSVCDSVSVWVGDKTTATDIKGKEVMVLGEVNINNSVFKQYFFETKCRDPNPVDSGCRGIDSKHWNSYCTTTHTFVFALTMDGKQAAWRFIRIDTACVCVLSRKAVRRA

SEQ ID NO: 13 - protein, 120 aa, Homo sapiens
SSSHPIFHRGEFSVCDSVSVWVGDKTTATDIKGKEVMVLGEVNINNSVFKQYFFETKCRDPNPVDSGCRGIDSKHWNSYCTTTHTFVLALTMDGKQAAWRFIRIDTACVCVLSRKAVRRA

SEQ ID NO: 14 - protein, 120 aa, Homo sapiens
SSSHPIFHRGEFSVCDSVSVWVGDKTTATDIKGKEVMVLGEVNINNSVFKQYFFETKCRDPNPVDSGCRGIDSKHWNSYCTTTHTFVEALTMDGKQAAWRFIRIDTACVCVLSRKAVRRA

SEQ ID NO: 15 - protein, 120 aa, Homo sapiens
SSSHPIFHRGEFSVCDSVSVWVGDKTTATDIKGKEVMVLGEVNINNSVFKQYFFETKCRDPNPVDSGCRGIDSKHWNSYCTTTHTFVGALTMDGKQAAWRFIRIDTACVCVLSRKAVRRA

SEQ ID NO: 16 - protein, 120 aa, Homo sapiens
SSSHPIFHRGEFSVCDSVSVWVGDKTTATDIKGKEVMVLGEVNINNSVFKQYFFETKCRDPNPVDSGCRGIDSKHWNSYCTTTHTFVKALTMDGKEAAWRFIRIDTACVCVLSRKAVRRA

SEQ ID NO: 17 - protein, 120 aa, Homo sapiens
SSSHPIFHRGEFSVCDSVSVWVGDKTTATDIKGKEVMVLGEVNINNSVFKQYFFETKCRDPNPVDSGCRGIDSKHWNSYCTTTHTFVKALTMDGKQAAWRFIRIDTACVCVLSVKAVRRA

SEQ ID NO: 18 - protein, 120 aa, Homo sapiens
SSSHPIFHRGEFSVCDSVSVWVGDKTTATDIKGKEVMVLGEVNINNSVFKQYFFETKCRDPNPVDSGCRGIDSKHWNSYCTTTHTFVKALTMDGKQAAWRFIRIDTACVCVLSFKAVRRA

SEQ ID NO: 19 - protein, 120 aa, Homo sapiens
SSSHPIFHRGEFSVCDSVSVWVGDKTTATDIKGKEVMVLGEVNINNSVFKQYFFETKCRDPNPVDSGCRGIDSKHWNSYCTTTHTFVKALTMDGKQAAWRFIRIDTACVCVLSGKAVRRA

SEQ ID NO: 20 - protein, 120 aa, Homo sapiens
SSSHPIFHRGEFSVCDSVSVWVGDKTTATDIKGKEVMVLGEVNINNSVFKQYFFETKCRDPNPVDSGCRGIDSKHWNSYCTTTHTFVKALTMDGKQAAWRFIRIDTACVCVLSLKAVRRA

SEQ ID NO: 21 - protein, 120 aa, Homo sapiens
SSSHPIFHRGEFSVCDSVSVWVGDKTTATDIKGKEVMVLGEVNINNSVFKQYFFETKCRDPNPVDSGCRGIDSKHWNSYCTTTHTFVKALTMDGKQAAWRAIRIDTACVCVLSRKAVRRA

SEQ ID NO: 22 - DNA, 726 bp, Homo sapiens
atgtccatgttgttctacactctgatcacagcttttctgatcggcatacaggcggaaccacactcagagagcaatgtccctgcaggacacaccatcccccaagcccactggactaaacttcagcattcccttgacactgcccttcgcagagcccgcagcgccccggcagcggcgatagctgcacgcgtggcggggcagacccgcaacattactgtggaccccaggctgtttaaaaagcggcgactccgttcaccccgtgtgctgtttagcacccagcctccccgtgaagctgcagacactcaggatctggacttcgaggtcggtggtgctgcccccttcaacaggactcacaggagcaagcggtcatcatcccatcccatcttccacaggggcgaagagtcggtgtgtgacagtgtcagcgtgtgggttggggataagaccaccgccacagacatcaagggcaaggaggtgatggtgttgggagaggtgaacattaacaacagtgtattcaaacagtacttttttgagaccaagtgccgggacccaaatcccgttgacagcgggtgccggggcattgactcaaagcactggaactcatattgtaccacgactcacacctttgtcaaggcgctgaccatggatggcaagcaggctgcctggcggtttatccggatagatacggcctgtgtgtgtgtgctcagcaggaaggctgtgagaagagcctga

SEQ ID NO: 23 - DNA, 726 bp, Homo sapiens
atgtccatgttgttctacactctgatcacagcttttctgatcggcatacaggcggaaccacactcagagagcaatgtccctgcaggacacaccatcccccaagcccactggactaaacttcagcattcccttgacactgcccttcgcagagcccgcagcgccccggcagcggcgatagctgcacgcgtggcggggcagacccgcaacattactgtggaccccaggctgtttaaaaagcggcgactccgttcaccccgtgtgctgtttagcacccagcctccccgtgaagctgcagacactcaggatctggacttcgaggtcggtggtgctgcccccttcaacaggactcacaggagcaagcggtcatcatcccatcccatcttccacaggggcgaattctcggtgtgtgacagtgtcagcgtgtgggttggggataagaccaccgccacagacatcgggggcaaggaggtgatggtgttgggagaggtgaacattaacaacagtgtattcaaacagtacttttttgagaccaagtgccgggacccaaatcccgttgacagcgggtgccggggcattgactcaaagcactggaactcatattgtaccacgactcacacctttgtcaaggcgctgaccatggatggcaagcaggctgcctggcggtttatccggatagatacggcctgtgtgtgtgtgctcagcaggaaggctgtgagaagagcctga

SEQ ID NO: 24 - DNA, 726 bp, Homo sapiens
atgtccatgttgttctacactctgatcacagcttttctgatcggcatacaggcggaaccacactcagagagcaatgtccctgcaggacacaccatcccccaagcccactggactaaacttcagcattcccttgacactgcccttcgcagagcccgcagcgccccggcagcggcgatagctgcacgcgtggcggggcagacccgcaacattactgtggaccccaggctgtttaaaaagcggcgactccgttcaccccgtgtgctgtttagcacccagcctccccgtgaagctgcagacactcaggatctggacttcgaggtcggtggtgctgcccccttcaacaggactcacaggagcaagcggtcatcatcccatcccatcttccacaggggcgaattctcggtgtgtgacagtgtcagcgtgtgggttggggataagaccaccgccacagacatcctgggcaaggaggtgatggtgttgggagaggtgaacattaacaacagtgtattcaaacagtacttttttgagaccaagtgccgggacccaaatcccgttgacagcgggtgccggggcattgactcaaagcactggaactcatattgtaccacgactcacacctttgtcaaggcgctgaccatggatggcaagcaggctgcctggcggtttatccggatagatacggcctgtgtgtgtgtgctcagcaggaaggctgtgagaagagcctga

SEQ ID NO: 25 - DNA, 726 bp, Homo sapiens
atgtccatgttgttctacactctgatcacagcttttctgatcggcatacaggcggaaccacactcagagagcaatgtccctgcaggacacaccatcccccaagcccactggactaaacttcagcattcccttgacactgcccttcgcagagcccgcagcgccccggcagcggcgatagctgcacgcgtggcggggcagacccgcaacattactgtggaccccaggctgtttaaaaagcggcgactccgttcaccccgtgtgctgtttagcacccagcctccccgtgaagctgcagacactcaggatctggacttcgaggtcggtggtgctgcccccttcaacaggactcacaggagcaagcggtcatcatcccatcccatcttccacaggggcgaattctcggtgtgtgacagtgtcagcgtgtgggttggggataagaccaccgccacagacatctacggcaaggaggtgatggtgttgggagaggtgaacattaacaacagtgtattcaaacagtacttttttgagaccaagtgccgggacccaaatcccgttgacagcgggtgccggggcattgactcaaagcactggaactcatattgtaccacgactcacacctttgtcaaggcgctgaccatggatggcaagcaggctgcctggcggtttatccggatagatacggcctgtgtgtgtgtgctcagcaggaaggctgtgagaagagcctga

SEQ ID NO: 26 - DNA, 726 bp, Homo sapiens
atgtccatgttgttctacactctgatcacagcttttctgatcggcatacaggcggaaccacactcagagagcaatgtccctgcaggacacaccatcccccaagcccactggactaaacttcagcattcccttgacactgcccttcgcagagcccgcagcgccccggcagcggcgatagctgcacgcgtggcggggcagacccgcaacattactgtggaccccaggctgtttaaaaagcggcgactccgttcaccccgtgtgctgtttagcacccagcctccccgtgaagctgcagacactcaggatctggacttcgaggtcggtggtgctgcccccttcaacaggactcacaggagcaagcggtcatcatcccatcccatcttccacaggggcgaattctcggtgtgtgacagtgtcagcgtgtgggttggggataagaccaccgccacagacatcaagggcaaggaggtgatggtgttgggagaggtgaacattaacaacagtgtattcaaacagtacttttttgagaccaagtgcctggacccaaatcccgttgacagcgggtgccggggcattgactcaaagcactggaactcatattgtaccacgactcacacctttgtcaaggcgctgaccatggatggcaagcaggctgcctggcggtttatccggatagatacggcctgtgtgtgtgtgctcagcaggaaggctgtgagaagagcctga

SEQ ID NO: 27 - DNA, 726 bp, Homo sapiens
atgtccatgttgttctacactctgatcacagcttttctgatcggcatacaggcggaaccacactcagagagcaatgtccctgcaggacacaccatcccccaagcccactggactaaacttcagcattcccttgacactgcccttcgcagagcccgcagcgccccggcagcggcgatagctgcacgcgtggcggggcagacccgcaacattactgtggaccccaggctgtttaaaaagcggcgactccgttcaccccgtgtgctgtttagcacccagcctccccgtgaagctgcagacactcaggatctggacttcgaggtcggtggtgctgcccccttcaacaggactcacaggagcaagcggtcatcatcccatcccatcttccacaggggcgaattctcggtgtgtgacagtgtcagcgtgtgggttggggataagaccaccgccacagacatcaagggcaaggaggtgatggtgttgggagaggtgaacattaacaacagtgtattcaaacagtacttttttgagaccaagtgcgcggacccaaatcccgttgacagcgggtgccggggcattgactcaaagcactggaactcatattgtaccacgactcacacctttgtcaaggcgctgaccatggatggcaagcaggctgcctggcggtttatccggatagatacggcctgtgtgtgtgtgctcagcaggaaggctgtgagaagagcctga

SEQ ID NO: 28 - DNA, 726 bp, Homo sapiens
atgtccatgttgttctacactctgatcacagcttttctgatcggcatacaggcggaaccacactcagagagcaatgtccctgcaggacacaccatcccccaagcccactggactaaacttcagcattcccttgacactgcccttcgcagagcccgcagcgccccggcagcggcgatagctgcacgcgtggcggggcagacccgcaacattactgtggaccccaggctgtttaaaaagcggcgactccgttcaccccgtgtgctgtttagcacccagcctccccgtgaagctgcagacactcaggatctggacttcgaggtcggtggtgctgcccccttcaacaggactcacaggagcaagcggtcatcatcccatcccatcttccacaggggcgaattctcggtgtgtgacagtgtcagcgtgtgggttggggataagaccaccgccacagacatcaagggcaaggaggtgatggtgttgggagaggtgaacattaacaacagtgtattcaaacagtacttttttgagaccaagtgccgggacccaaatcccgttgccagcgggtgccggggcattgactcaaagcactggaactcatattgtaccacgactcacacctttgtcaaggcgctgaccatggatggcaagcaggctgcctggcggtttatccggatagatacggcctgtgtgtgtgtgctcagcaggaaggctgtgagaagagcctga

SEQ ID NO: 29 - DNA, 726 bp, Homo sapiens
atgtccatgttgttctacactctgatcacagcttttctgatcggcatacaggcggaaccacactcagagagcaatgtccctgcaggacacaccatcccccaagcccactggactaaacttcagcattcccttgacactgcccttcgcagagcccgcagcgccccggcagcggcgatagctgcacgcgtggcggggcagacccgcaacattactgtggaccccaggctgtttaaaaagcggcgactccgttcaccccgtgtgctgtttagcacccagcctccccgtgaagctgcagacactcaggatctggacttcgaggtcggtggtgctgcccccttcaacaggactcacaggagcaagcggtcatcatcccatcccatcttccacaggggcgaattctcggtgtgtgacagtgtcagcgtgtgggttggggataagaccaccgccacagacatcaagggcaaggaggtgatggtgttgggagaggtgaacattaacaacagtgtattcaaacagtacttttttgagaccaagtgccgggacccaaatcccgttggcagcgggtgccggggcattgactcaaagcactggaactcatattgtaccacgactcacacctttgtcaaggcgctgaccatggatggcaagcaggctgcctggcggtttatccggatagatacggcctgtgtgtgtgtgctcagcaggaaggctgtgagaagagcctga

SEQ ID NO: 30 - DNA, 726 bp, Homo sapiens
atgtccatgttgttctacactctgatcacagcttttctgatcggcatacaggcggaaccacactcagagagcaatgtccctgcaggacacaccatcccccaagcccactggactaaacttcagcattcccttgacactgcccttcgcagagcccgcagcgccccggcagcggcgatagctgcacgcgtggcggggcagacccgcaacattactgtggaccccaggctgtttaaaaagcggcgactccgttcaccccgtgtgctgtttagcacccagcctccccgtgaagctgcagacactcaggatctggacttcgaggtcggtggtgctgcccccttcaacaggactcacaggagcaagcggtcatcatcccatcccatcttccacaggggcgaattctcggtgtgtgacagtgtcagcgtgtgggttggggataagaccaccgccacagacatcaagggcaaggaggtgatggtgttgggagaggtgaacattaacaacagtgtattcaaacagtacttttttgagaccaagtgccgggacccaaatcccgttgacagcgggtgccggggcattgactcattgcactggaactcatattgtaccacgactcacacctttgtcaaggcgctgaccatggatggcaagcaggctgcctggcggtttatccggatagatacggcctgtgtgtgtgtgctcagcaggaaggctgtgagaagagcctga

SEQ ID NO: 31 - DNA, 726 bp, Homo sapiens
atgtccatgttgttctacactctgatcacagcttttctgatcggcatacaggcggaaccacactcagagagcaatgtccctgcaggacacaccatcccccaagcccactggactaaacttcagcattcccttgacactgcccttcgcagagcccgcagcgccccggcagcggcgatagctgcacgcgtggcggggcagacccgcaacattactgtggaccccaggctgtttaaaaagcggcgactccgttcaccccgtgtgctgtttagcacccagcctccccgtgaagctgcagacactcaggatctggacttcgaggtcggtggtgctgcccccttcaacaggactcacaggagcaagcggtcatcatcccatcccatcttccacaggggcgaattctcggtgtgtgacagtgtcagcgtgtgggttggggataagaccaccgccacagacatcaagggcaaggaggtgatggtgttgggagaggtgaacattaacaacagtgtattcaaacagtacttttttgagaccaagtgccgggacccaaatcccgttgacagcgggtgccggggcattgactcaaagcactggaactcatattgtaccacgactcacacctttgtctttgcgctgaccatggatggcaagcaggctgcctggcggtttatccggatagatacggcctgtgtgtgtgtgctcagcaggaaggctgtgagaagagcctga

SEQ ID NO: 32 - DNA, 726 bp, Homo sapiens
atgtccatgttgttctacactctgatcacagcttttctgatcggcatacaggcggaaccacactcagagagcaatgtccctgcaggacacaccatcccccaagcccactggactaaacttcagcattcccttgacactgcccttcgcagagcccgcagcgccccggcagcggcgatagctgcacgcgtggcggggcagacccgcaacattactgtggaccccaggctgtttaaaaagcggcgactccgttcaccccgtgtgctgtttagcacccagcctccccgtgaagctgcagacactcaggatctggacttcgaggtcggtggtgctgcccccttcaacaggactcacaggagcaagcggtcatcatcccatcccatcttccacaggggcgaattctcggtgtgtgacagtgtcagcgtgtgggttggggataagaccaccgccacagacatcaagggcaaggaggtgatggtgttgggagaggtgaacattaacaacagtgtattcaaacagtacttttttgagaccaagtgccgggacccaaatcccgttgacagcgggtgccggggcattgactcaaagcactggaactcatattgtaccacgactcacacctttgtcttggcgctgaccatggatggcaagcaggctgcctggcggtttatccggatagatacggcctgtgtgtgtgtgctcagcaggaaggctgtgagaagagcctga

SEQ ID NO: 33 - DNA, 726 bp, Homo sapiens
atgtccatgttgttctacactctgatcacagcttttctgatcggcatacaggcggaaccacactcagagagcaatgtccctgcaggacacaccatcccccaagcccactggactaaacttcagcattcccttgacactgcccttcgcagagcccgcagcgccccggcagcggcgatagctgcacgcgtggcggggcagacccgcaacattactgtggaccccaggctgtttaaaaagcggcgactccgttcaccccgtgtgctgtttagcacccagcctccccgtgaagctgcagacactcaggatctggacttcgaggtcggtggtgctgcccccttcaacaggactcacaggagcaagcggtcatcatcccatcccatcttccacaggggcgaattctcggtgtgtgacagtgtcagcgtgtgggttggggataagaccaccgccacagacatcaagggcaaggaggtgatggtgttgggagaggtgaacattaacaacagtgtattcaaacagtacttttttgagaccaagtgccgggacccaaatcccgttgacagcgggtgccggggcattgactcaaagcactggaactcatattgtaccacgactcacacctttgtcgaggcgctgaccatggatggcaagcaggctgcctggcggtttatccggatagatacggcctgtgtgtgtgtgctcagcaggaaggctgtgagaagagcctga

SEQ ID NO: 34 - DNA, 726 bp, Homo sapiens
atgtccatgttgttctacactctgatcacagcttttctgatcggcatacaggcggaaccacactcagagagcaatgtccctgcaggacacaccatcccccaagcccactggactaaacttcagcattcccttgacactgcccttcgcagagcccgcagcgccccggcagcggcgatagctgcacgcgtggcggggcagacccgcaacattactgtggaccccaggctgtttaaaaagcggcgactccgttcaccccgtgtgctgtttagcacccagcctccccgtgaagctgcagacactcaggatctggacttcgaggtcggtggtgctgcccccttcaacaggactcacaggagcaagcggtcatcatcccatcccatcttccacaggggcgaattctcggtgtgtgacagtgtcagcgtgtgggttggggataagaccaccgccacagacatcaagggcaaggaggtgatggtgttgggagaggtgaacattaacaacagtgtattcaaacagtacttttttgagaccaagtgccgggacccaaatcccgttgacagcgggtgccggggcattgactcaaagcactggaactcatattgtaccacgactcacacctttgtcggggcgctgaccatggatggcaagcaggctgcctggcggtttatccggatagatacggcctgtgtgtgtgtgctcagcaggaaggctgtgagaagagcctga

SEQ ID NO: 35 - DNA, 726 bp, Homo sapiens
atgtccatgttgttctacactctgatcacagcttttctgatcggcatacaggcggaaccacactcagagagcaatgtccctgcaggacacaccatcccccaagcccactggactaaacttcagcattcccttgacactgcccttcgcagagcccgcagcgccccggcagcggcgatagctgcacgcgtggcggggcagacccgcaacattactgtggaccccaggctgtttaaaaagcggcgactccgttcaccccgtgtgctgtttagcacccagcctccccgtgaagctgcagacactcaggatctggacttcgaggtcggtggtgctgcccccttcaacaggactcacaggagcaagcggtcatcatcccatcccatcttccacaggggcgaattctcggtgtgtgacagtgtcagcgtgtgggttggggataagaccaccgccacagacatcaagggcaaggaggtgatggtgttgggagaggtgaacattaacaacagtgtattcaaacagtacttttttgagaccaagtgccgggacccaaatcccgttgacagcgggtgccggggcattgactcaaagcactggaactcatattgtaccacgactcacacctttgtcaaggcgctgaccatggatggcaaggaggctgcctggcggtttatccggatagatacggcctgtgtgtgtgtgctcagcaggaaggctgtgagaagagcctga

SEQ ID NO: 36 - DNA, 726 bp, Homo sapiens
atgtccatgttgttctacactctgatcacagcttttctgatcggcatacaggcggaaccacactcagagagcaatgtccctgcaggacacaccatcccccaagcccactggactaaacttcagcattcccttgacactgcccttcgcagagcccgcagcgccccggcagcggcgatagctgcacgcgtggcggggcagacccgcaacattactgtggaccccaggctgtttaaaaagcggcgactccgttcaccccgtgtgctgtttagcacccagcctccccgtgaagctgcagacactcaggatctggacttcgaggtcggtggtgctgcccccttcaacaggactcacaggagcaagcggtcatcatcccatcccatcttccacaggggcgaattctcggtgtgtgacagtgtcagcgtgtgggttggggataagaccaccgccacagacatcaagggcaaggaggtgatggtgttgggagaggtgaacattaacaacagtgtattcaaacagtacttttttgagaccaagtgccgggacccaaatcccgttgacagcgggtgccggggcattgactcaaagcactggaactcatattgtaccacgactcacacctttgtcaaggcgctgaccatggatggcaagcaggctgcctggcggtttatccggatagatacggcctgtgtgtgtgtgctcagcgtcaaggctgtgagaagagcctga

SEQ ID NO: 37 - DNA, 726 bp, Homo sapiens
atgtccatgttgttctacactctgatcacagcttttctgatcggcatacaggcggaaccacactcagagagcaatgtccctgcaggacacaccatcccccaagcccactggactaaacttcagcattcccttgacactgcccttcgcagagcccgcagcgccccggcagcggcgatagctgcacgcgtggcggggcagacccgcaacattactgtggaccccaggctgtttaaaaagcggcgactccgttcaccccgtgtgctgtttagcacccagcctccccgtgaagctgcagacactcaggatctggacttcgaggtcggtggtgctgcccccttcaacaggactcacaggagcaagcggtcatcatcccatcccatcttccacaggggcgaattctcggtgtgtgacagtgtcagcgtgtgggttggggataagaccaccgccacagacatcaagggcaaggaggtgatggtgttgggagaggtgaacattaacaacagtgtattcaaacagtacttttttgagaccaagtgccgggacccaaatcccgttgacagcgggtgccggggcattgactcaaagcactggaactcatattgtaccacgactcacacctttgtcaaggcgctgaccatggatggcaagcaggctgcctggcggtttatccggatagatacggcctgtgtgtgtgtgctcagcttcaaggctgtgagaagagcctga

SEQ ID NO: 38 - DNA, 726 bp, Homo sapiens
atgtccatgttgttctacactctgatcacagcttttctgatcggcatacaggcggaaccacactcagagagcaatgtccctgcaggacacaccatcccccaagcccactggactaaacttcagcattcccttgacactgcccttcgcagagcccgcagcgccccggcagcggcgatagctgcacgcgtggcggggcagacccgcaacattactgtggaccccaggctgtttaaaaagcggcgactccgttcaccccgtgtgctgtttagcacccagcctccccgtgaagctgcagacactcaggatctggacttcgaggtcggtggtgctgcccccttcaacaggactcacaggagcaagcggtcatcatcccatcccatcttccacaggggcgaattctcggtgtgtgacagtgtcagcgtgtgggttggggataagaccaccgccacagacatcaagggcaaggaggtgatggtgttgggagaggtgaacattaacaacagtgtattcaaacagtacttttttgagaccaagtgccgggacccaaatcccgttgacagcgggtgccggggcattgactcaaagcactggaactcatattgtaccacgactcacacctttgtcaaggcgctgaccatggatggcaagcaggctgcctggcggtttatccggatagatacggcctgtgtgtgtgtgctcagcgggaaggctgtgagaagagcctga

SEQ ID NO: 39 - DNA, 726 bp, Homo sapiens
atgtccatgttgttctacactctgatcacagcttttctgatcggcatacaggcggaaccacactcagagagcaatgtccctgcaggacacaccatcccccaagcccactggactaaacttcagcattcccttgacactgcccttcgcagagcccgcagcgccccggcagcggcgatagctgcacgcgtggcggggcagacccgcaacattactgtggaccccaggctgtttaaaaagcggcgactccgttcaccccgtgtgctgtttagcacccagcctccccgtgaagctgcagacactcaggatctggacttcgaggtcggtggtgctgcccccttcaacaggactcacaggagcaagcggtcatcatcccatcccatcttccacaggggcgaattctcggtgtgtgacagtgtcagcgtgtgggttggggataagaccaccgccacagacatcaagggcaaggaggtgatggtgttgggagaggtgaacattaacaacagtgtattcaaacagtacttttttgagaccaagtgccgggacccaaatcccgttgacagcgggtgccggggcattgactcaaagcactggaactcatattgtaccacgactcacacctttgtcaaggcgctgaccatggatggcaagcaggctgcctggcggtttatccggatagatacggcctgtgtgtgtgtgctcagcttgaaggctgtgagaagagcctga

SEQ ID NO: 40 - DNA, 726 bp, Homo sapiens
atgtccatgttgttctacactctgatcacagcttttctgatcggcatacaggcggaaccacactcagagagcaatgtccctgcaggacacaccatcccccaagcccactggactaaacttcagcattcccttgacactgcccttcgcagagcccgcagcgccccggcagcggcgatagctgcacgcgtggcggggcagacccgcaacattactgtggaccccaggctgtttaaaaagcggcgactccgttcaccccgtgtgctgtttagcacccagcctccccgtgaagctgcagacactcaggatctggacttcgaggtcggtggtgctgcccccttcaacaggactcacaggagcaagcggtcatcatcccatcccatcttccacaggggcgaattctcggtgtgtgacagtgtcagcgtgtgggttggggataagaccaccgccacagacatcaagggcaaggaggtgatggtgttgggagaggtgaacattaacaacagtgtattcaaacagtacttttttgagaccaagtgccgggacccaaatcccgttgacagcgggtgccggggcattgactcaaagcactggaactcatattgtaccacgactcacacctttgtcaaggcgctgaccatggatggcaagcaggctgcctggcgggcgatccggatagatacggcctgtgtgtgtgtgctcagcaggaaggctgtgagaagagcctga

SEQ ID NO: 41 - DNA, 19 bp, Artificial sequence
  primer F
ggaattcatgtccatgttg

SEQ ID NO: 42 - DNA, 19 bp, Artificial sequence
  primer R
caagctttcaggctcttct

(19) United States
(12) Patent Application Publication
(10) Pub. No.: US 2022/0088139 A1
(43) Pub. Date: Mar. 24, 2022

(54) NERVE GROWTH FACTOR MUTANT

(71) Applicant: Staidson (Beijing) Biopharmaceuticals Co., Ltd., Beijing (CN)

(72) Inventors: Chao Wang, Beijing (CN); Jiabin Li, Beijing (CN); Qing Zhu, Beijing (CN); Jiala Li, Beijing (CN)

(21) Appl. No.: 17/455,880

(22) Filed: Nov. 19, 2021

Related U.S. Application Data
(63) Continuation-in-part of application No. 16/085,977, filed on Sep. 17, 2018, now Pat. No. 11,213,568.

(30) Foreign Application Priority Data
Mar. 18, 2016 (CN) 201610159303.7

(51) Int. Cl. A61K 38/18; A61P 27/02; A61P 25/28

(52) U.S. Cl. CPC A61K 38/185; A61P 25/28; A61P 27/02

(57) ABSTRACT

Provided is a nerve growth factor mutant, wherein the nerve growth factor mutant is an amino acid sequence as shown by any one of SEQ ID No: 3 to SEQ ID No: 21 in the sequence listing. The advantage of the nerve growth factor mutant lies in that the mutation of a nerve growth factor can alleviate side effects such as pain, falling within the field of biological pharmacy.

### CROSS-REFERENCE TO RELATED APPLICATION

This application is a continuation-in-part of the U.S. patent application Ser. No. 16/085,977, filed on Sep. 17, 2018, which is the U.S. National Phase Application of PCT International Application Number PCT/CN2017/077038, filed on Mar. 17, 2017, designating the United States of America and published in the Chinese language, which is an International Application of and claims the benefit of priority to Chinese Patent Application No. 201610159303.7, filed on Mar. 18, 2016. The disclosures of the above-referenced applications are hereby expressly incorporated by reference in their entireties.

### REFERENCE TO SEQUENCE LISTING

A Sequence Listing submitted as an ASCII text file via EFS-Web is hereby incorporated by reference in accordance with 35 U.S.C. § 1.52(e). The name of the ASCII text file for the Sequence Listing is SeqList-DRGN005-002P1.txt, the date of creation of the ASCII text file is Nov. 19, 2021, and the size of the ASCII text file is 43 KB.

### TECHNICAL FIELD

The present disclosure relates to a nerve growth factor mutant, and belongs to the field of biopharmaceuticals.

### BACKGROUND

Pain may be divided into two types: sensory pain and neuropathic pain according to its neurophysiological mechanism. The former is directly caused by noxious stimulation, relates to tissue damage or inflammatory reaction, and is also known as inflammatory pain. The latter is a chronic pain directly caused by the damage or disease of somatosensory nervous system.

Nerve Growth Factor (NGF) is the first neurotrophic factor discovered in mouse sarcoma cells by Italian scientist Levi-Montlcini in 1953. NGF is a neuronal growth regulator having a dual biological function of neuron nutrition and promoting neurite growth, which plays an important regulatory role in the development, differentiation, growth, regeneration, and expression of functional properties of central and peripheral neurons. NGF includes three subunits of α, β, and γ. The β subunit is an active region, which is formed by combining two single chains through a non-covalent bond. Levi-Montalcini won the Nobel Prize for discovering NGF. At present, a number of NGF products have been marketed at home and abroad, and are mainly used for the treatment of nervous system dysplasia, including amblyopia, neuroma, various nerve injuries and nervous system diseases.

NGF is present in a variety of species and is abundant in male mouse submandibular gland, bovine seminal plasma, snake venom, guinea pig prostate, and human placental tissue. The amino acid sequence homology between mouse NGF and human NGF is up to 90%. In consideration of the species diversity of mouse NGF applied to human body, the risk of the potential pathogenic factor of a mouse as a raw material and the limitation of human placenta tissue raw material, there is a very good application prospect for the development of the genetic engineering technology for preparing recombinant human NGF (rhNGF) to replace the extracted mouse NGF and human NGF.

Mature NGF in vivo exists as a homodimer, in which each peptide chain includes 120 amino acids. The human NGF gene is located on a short arm of chromosome 1, and the complete NGF exon consists of 241 amino acids, commonly referred to as a prepro NGF precursor. The signal peptide of the prepro NGF precursor in the endoplasmic reticulum is cleaved to form a pro NGF precursor (223 amino acids). The pro NGF precursor exists in a form of a homodimer in the endoplasmic reticulum, and then transferred to a golgi apparatus, in which the precursor undergoes digestion with Furin to form a mature NGF dimer (the monomer contains 120 amino acids) which is then transported outside the cell. Meanwhile, some uncleaved pro NGF precursors are secreted outside the cell.

Recombinant human NGF avoids some potential pathogenic risks, but there are still major problems in actual application: 1) as for maintaining the biological activity of NGF, like other proteins, the biological activity of NGF depends on the secondary and tertiary structures thereof, and thus it is particularly important to maintain its biological activity during preparation, purification, storage and administration; and 2) NGF may cause serious pain, which cannot be tolerated by some patients, during application, thereby partially limiting its use. NGF is involved in the pathophysiological process of pain through affecting the release of inflammatory mediators, the opening of ion channels, and promoting the growth of nerve fibers to cause pain; and involved in the development of pain through regulating ion channels and molecular signals. Some scholars speculate that NGF may also cause pain through promoting the expression of pain-inducing substances, and may change the budding and regeneration of neurons after injury of organism. Current research found that the maximum dose that does not cause hyperalgesia in humans is 0.03 μg/kg (Petty et al., 1994-29). However, such low dose limits the application of NGF and also limits the expansion of its indications, such as use for the central nervous system.

Therefore, in order to avoid the above problems, it is necessary to seek a recombinant hNGF capable of alleviating side effects such as pain or even painlessness, thereby increasing the dosage and the subjects, and providing the possibility to expand the indications and apply to the central nervous system.

### SUMMARY

An object of the present disclosure is to provide a series of nerve growth factor mutants, i.e., recombinant hNGFs, which are capable of alleviating side effects such as pain and are even painless, More preferably, provided is a series of nerve growth factor mutants, which have a high biological activity and are capable of alleviating side effects such as pain or even painless. To achieve the above object, the present disclosure adopts the following technical solutions.

Provided is a nerve growth factor mutant, in which mutation sites of the mutant include: Phe12Glu, Lys32Gly, Lys32Leu, Lys32Tyr, Arg59Leu, Arg59Ala, Asp65Ala, Asp65Gly, Lys74Leu, Lys88Phe Lys88Leu, Lys88Glu, Lys88Gly, Gln96Glu, Arg114Val, Arg114Phe, Arg114Gly, Arg114Leu, Phe101Ala, or any combination of the above mutation sites with respect to a parental nerve growth factor; preferably with respect to a parental human nerve growth factor; and preferably with respect to a parental wild-type human nerve growth factor.

The nerve growth factor mutant has an amino acid sequence of any one of SEQ ID No: 3 to SEQ ID No: 21 in the sequence listing.

Provided is a nucleotide sequence, encoding the nerve growth factor mutant.

The nucleotide sequence is a nucleotide sequence of SEQ ID No: 22 to SEQ ID No: 40 in the sequence listing.

The recombinant hNGF mutant of the present disclosure is obtained through single point mutation based on the wild-type hNGF sequence, and the amino acid sequences thereof and the corresponding nucleotide sequences encoding them are shown as follows:

Phe12Glu: having an amino acid sequence as shown in SEQ ID No: 3, encoded by a nucleotide sequence as shown in SEQ ID No: 22;

Lys32Gly: having an amino acid sequence as shown in SEQ ID No: 4, encoded by a nucleotide sequence as shown in SEQ ID No: 23;

Lys32Leu: having an amino acid sequence as shown in SEQ ID No: 5, encoded by a nucleotide sequence as shown in SEQ ID No: 24;

Lys32Tyr: having an amino acid sequence as shown in SEQ ID No: 6, encoded by a nucleotide sequence as shown in SEQ ID No: 25;

Arg59Leu: having an amino acid sequence as shown in SEQ ID No: 7, encoded by a nucleotide sequence as shown in SEQ ID No: 26;

Arg59Ala: having an amino acid sequence as shown in SEQ ID No: 8, encoded by a nucleotide sequence as shown in SEQ ID No: 27;

Asp65Ala: having an amino acid sequence as shown in SEQ ID No: 9, encoded by a nucleotide sequence as shown in SEQ ID No: 28;

Asp65Gly: having an amino acid sequence as shown in SEQ ID No: 10, encoded by a nucleotide sequence as shown in SEQ ID No: 29;

Lys74Leu: having an amino acid sequence as shown in SEQ ID No: 11, encoded by a nucleotide sequence as shown in SEQ ID No: 30;

Lys88Phe: having an amino acid sequence as shown in SEQ ID No: 12, encoded by a nucleotide sequence as shown in SEQ ID No: 31;

Lys88Leu: having an amino acid sequence as shown in SEQ ID No: 13, encoded by a nucleotide sequence as shown in SEQ ID No: 32;

Lys88Glu: having an amino acid sequence as shown in SEQ ID No: 14, encoded by a nucleotide sequence as shown in SEQ ID No: 33;

Lys88Gly: having an amino acid sequence as shown in SEQ ID No: 15, encoded by a nucleotide sequence as shown in SEQ ID No: 34;

Gln96Glu: having an amino acid sequence as shown in SEQ ID No: 16, encoded by a nucleotide sequence as shown in SEQ ID No: 35;

Arg114Val: having an amino acid sequence as shown in SEQ ID No: 17, encoded by a nucleotide sequence as shown in SEQ ID No: 36;

Arg114Phe: having an amino acid sequence as shown in SEQ ID No: 18, encoded by a nucleotide sequence as shown in SEQ ID No: 37;

Arg114Gly: having an amino acid sequence as shown in SEQ ID No: 19, encoded by a nucleotide sequence as shown in SEQ ID No: 38;

Arg114Leu: having an amino acid sequence as shown in SEQ ID No: 20, encoded by a nucleotide sequence as shown in SEQ ID No: 39; and

Phe101Ala: having an amino acid sequence as shown in SEQ ID No: 21, encoded by a nucleotide sequence as shown in SEQ ID No: 40.

Provided is a long-acting nerve growth factor mutant, in which the long-acting nerve growth factor mutant is obtained from any one of the above amino acid sequences.

Preferably, the long-acting nerve growth factor mutant is obtained through chemical modification, and preferably, the long-acting nerve growth factor mutant is a conjugate of polyethylene glycol with a nerve growth factor mutant.

Preferably, the long-acting nerve growth factor mutant is a fusion protein obtained by fusing with an other protein. Preferably, the other protein is a human albumin, a human albumin analog, a fragment of a human albumin, an Fc moiety of an immunoglobulin, an analog of an Fc moiety of an immunoglobulin, or a fragment of an Fc moiety of an immunoglobulin.

Preferably, the fusion protein is obtained by fusing a C-terminal of the long-acting nerve growth factor mutant with an N-terminal of an albumin or an Fc protein directly or via a peptide linker.

Provided is an expression vector, including the nucleotide sequence.

The expression vector is selected from the group consisting of a DNA vector and a virus vector.

The DNA vector is selected from the group consisting of a DNA plasmid vector, a liposome bound thereto, a molecular conjugate bound thereto, and a polymer bound thereto, and preferably, the DNA plasmid vector is a eukaryotic expression vector; and the virus vector is selected from the group consisting of an adeno-associated virus vector, a lentivirus vector and an adenovirus vector.

Provided is a method for expressing the expression vector, including: transfecting the expression vector into a host cell, and culturing the resulting recombinant cell to express the expression vector, so as to obtain the nerve growth factor mutant.

Provided is a host cell, including the expression vector.

The host cell is a mammalian cell.

The mammalian cell is a Chinese hamster ovary cell, a human embryonic kidney 293 cell, a COS cell or a Hela cell.

Provided is a pharmaceutical composition, including a pharmaceutically acceptable excipient, and one or more of the above-mentioned nerve growth factor mutant, the above-mentioned expression vector, and the above-mentioned host cell.

The medicament of the present disclosure may be prepared into various forms such as an injection, a capsule, a tablet or powder, and medicaments having the above various dosage forms may be prepared according to a conventional method in the field of pharmacy.

The pharmaceutical composition is preferably an injection including a pharmaceutically acceptable excipient and the above-mentioned nerve growth factor mutant.

If necessary, one or more pharmaceutically acceptable carriers may be further added to the above pharmaceutical composition, and the carrier includes conventional diluents, stabilizers, surfactants, preservatives and the like in the pharmaceutical field.

Provided is use of the nerve growth factor mutant in the preparation of a medicament fortreating a nervous system disease. The nervous system disease refers to a disease associated with neuronal degeneration or injury in the central and/or peripheral nervous system. Specific examples of the nervous system disease include, but are not limited to, Alzheimer's disease, Parkinson's disease, Huntington's disease, stroke, ALS, peripheral neuropathy, and other disorders characterized by necrosis or loss of neuron regardless central neuron, peripheral neuron, or motorneuron, except treating nerve damage caused by trauma, burns, kidney failure, or injury. For example, peripheral neuropathy associated with certain disorders is such as a neuropathy associated with diabetes, AIDS, chemotherapy or neurotrophic keratitis.

Provided is a method of using a nerve growth factor mutant to treat or inhibit a disease associated with neuronal degeneration or injury in the central and/or peripheral nervous system comprising: administering the nerve growth factor to a subject in need thereof. Specific examples of the nervous system disease include, but are not limited to, Alzheimer's disease, Parkinson's disease, Huntington's disease, stroke, ALS, peripheral neuropathy, and other disorders characterized by necrosis or loss of neuron regardless central neuron, peripheral neuron, or motorneuron, except treating nerve damage caused by trauma, burns, kidney failure, or injury. For example, peripheral neuropathy associated with certain disorders is such as a neuropathy associated with diabetes, AIDS, chemotherapy or neurotrophic keratitis.

Preferably, the disease associated with neuronal degeneration or injury in the central and/or peripheral nervous system selected from the group consisting of: diabetic neuropathy, Alzheimer's disease, or neurotrophic keratitis.

Preferably, the nerve growth factor mutant comprising Phe12Glu with reference to the amino acid positions set forth in a wild-type human nerve growth factor.

Preferably, using the nerve growth factor mutant to improve a subject in one or more neurological diseases: diabetic neuropathy, Alzheimer's disease or neurotrophic keratitis. Preferably, using the nerve growth factor mutant to improve wound healing in diabeties, improve the spatial cognition and the ability of learning and memory of Alzheimer's subject, restore the integrity of subject's cornea, or improve the damage of corneal nerve caused by neurotrophic keratitis.

The medicament for treating a nervous system disease prepared by a nerve growth factor mutant may be administered to a patient. The exact dosage will depend on the disease to be treated, and may be determined by one skilled in the art using known techniques. Additionally, as is known in the art, an adjustment needs to be made based on age, weight, general health, sex, diet, time of administration, drug interaction, and severity of the disease, and this may be determined by one skilled in the art through routine experimentation. The patient mentioned herein includes human, and other animals and organisms. Therefore, these methods may be used for treating human and livestock.

The administration of the medicament for treating a nervous system disease prepared by the nerve growth factor mutant of the present disclosure may be carried out by various methods, including, but not limited to, oral, subcutaneous, intravenous, intracerebral, intranasal, transdermal, intraperitoneal, intramuscular, intrapulmonary, vaginal, rectal, and intraocular administrations. Under some circumstances, such as treating a wound, it may be applied directly in a form of a solution or spray.

The pharmaceutical composition of the present disclosure includes the nerve growth factor mutant in a form suitable for administration to a patient. In a preferred example, the pharmaceutical composition is in a water soluble form, and may include, for example, a carrier, a excipient, a stabilizer, a buffer, a salt, an antioxidant, a hydrophilic polymer, an amino acid, a carbohydrate, an ionic or nonionic surfactant, polyethylene glycol, propylene glycol or the like. The medicament prepared by the nerve growth factor mutant may also be implanted in a sustained release form by techniques known in the art or embedded in a microcapsule form.

Provided is use of the nerve growth factor mutant in the preparation of a medicament for effectively reducing weight.

Provided is use of the nerve growth factor mutant in the preparation of a long-acting nerve growth factor.

Advantages of the present disclosure are shown as follows. As compared with the wild-type nerve growth factor in the prior art, the present disclosure may alleviate side effects such as pain and even be painless, and further, the biological activity of part of the nerve growth factor mutants may be significantly improved.

The present disclosure will be further described hereinafter in conjunction with drawings and specific embodiments, which are not intended to limit the scope of the present disclosure. All equivalent substitutions in the art in accordance with the present disclosure fall into the scope of the present disclosure.

### BRIEF DESCRIPTION OF THE DRAWINGS

FIG. 1 is a result of the SDS PAGE electrophoresis of the wild-type hNGF purified by Superdex 75 column in Example 4.

FIG. 2(A) and FIG. 2(B) are results of the activity measurement of the mutants in Example 5.

FIG. 3 (A) and FIG. 3 (B) are results of the pain threshold measurement of the short-term administrated to mice in Example 7.

FIG. 4 (A), FIG. 4 (B) and FIG. 4 (C) are results of the pain threshold measurement of the long-term administrated to mice in Example 7.

FIG. 5 is a result of the behavioral experiments of the mice injected with a mutant and a wild-type hNGF respectively in Example 8 by observing the leg lifting maintenance time.

FIG. 6 is the result of depicts wound closure rate of diabetic mice treated with NGF (SuTaiSheng® mouse NGF or Phe12Glu). PBS treatment served as negative control.

FIGS. 7A and 7B depict corneal fluorescein sodium staining score (FIG. 7A) and average corneal nerve length (FIG. 7B) of rat models of neurotrophic keratitis treated with NGF (SuTaiSheng® mouse NGF or Phe12Glu), or 0.9% sodium chloride solution as negative control.

### DETAILED DESCRIPTION

### Example 1: Plasmid Construction of Wild-Type hNGF and its Mutants

1. Construction of Expression Plasmid Containing DNA Sequence of Wild-Type hNGF

The DNA sequence of wild-type hNGF was synthesized (SEQ ID NO: 1 in the sequence listing), and the target sequence was amplified by PCR using primers (F: GGAATTCATGTCCATGTTG (SEQ ID NO: 41), R: CAAGCTTTCAGGCTCTTCT (SEQ ID NO: 42)). The PCR product was digested with EcorI (NEB #R0101S), and then the resulting digested product was subjected to a secondary digestion with Hind III (NEB #R0104S). The pcDNA3.1(−) expression vector was digested in the same manner. The digested vector and the fragments amplified by PCR were subjected to agarose gel electrophoresis. The target fragments were cleaved, and the digested vector and the target DNA fragments were respectively recovered by using a DNA gel recovery kit (TIANGEN, #DP209-03) and were ligated by a DNA ligase kit (Takara/6022) at 16° C. for 1 h, to complete the plasmid construction of the wild-type hNGF.

2. Construction of Expression Plasmid Containing DNA Sequence of hNGF Mutants

In the same manner as the above, the plasmids of all mutants were synthesized and constructed. The DNA sequences of the mutants were nucleotide sequences from SEQ ID No: 22 to SEQ ID No: 40 in the sequence listing.

### Example 2: Transformation and Extraction of Plasmids Containing hNGF and its Mutants

1. Transformation

The plasmids containing hNGF and its mutants constructed in the above Example 1 were subjected to a heat shock transformation. The top 10 competent cells (Tiangen/CB104-02) were taken out from the −70° C. refrigerator and immediately thawed on ice, and 50 μl of competent cells were taken for transformation. 2 μl of the plasmid was added to the 50 μl of competent cells, mixed by flicking, subjected to an ice bath for 30 min, and then subjected to a dry bath at 42° C. for 90 s, during which the centrifuge tube was not shaken, and the centrifuge tube was immediately placed on ice for 2 min after taking out of the dry bath. 500 μl of antibody-free LB (Luria-Bertani)/SOC (Super Optimal broth with Catabolite repression) medium was added, and cultured at 37° C. for 45 min on a shaker at 150 rpm/min. All the liquid in the centrifuge tube was poured onto the LB plate and spread evenly. The plate, after drying, was inverted in an incubator for 16 h.

2. Large Scale Extraction of Plasmid

The single colonies obtained in the above 2.1 experiment were picked up, inoculated into 500 μl of LB liquid medium and cultured at 37° C. for 7 h, and the bacterial solution was sent for sequencing. The correct bacterial solution confirmed by sequencing was subjected to a lot of shaking, and 500 μl of the bacterial solution was inoculated into 500 ml of LB medium and cultured at 37° C. for 16 h. The overnight cultured solution was collected by centrifugation at 4° C., centrifuged at 6000×g for 10 min and the supernatant was completely discarded. The plasmid was large-scale extracted by using a Plasmid Maxi Kit (purchased from QIAGEN, Cat. No. 12163), and the concentration was measured for use.

### Example 3: Expression of Wild-Type hNGF and its Mutants

The wild-type hNGF and its mutants plasmids large-scale extracted in the above Example 2 were transfected into 293F cells, and the expression supernatant was collected and quantified on day 4 after transfection.

### Experimental Procedures

1. One day before transfection, 900 ml of 293F cells in total were inoculated at 0.5×10⁶/ml in 300 ml/bottle.

2. Cells were counted on the day of the transfection, and the cell density was about 1.0×10⁶/ml with a viability of 99% or more.

3. Transfection: 36 ml of a cell culture medium was taken into an 125 ml culture flask; 360 ug of plasmid was added and mixed evenly; and then 1080 ug of PEI was added and mix evenly, leave it to stand at room temperature for 15 min, mixed with cells at about 12.3 ml/bottle, and incubated at 37° C., in 8% CO₂, under 120 RPM.

4. On the fourth day after the transfection, the cell supernatant was collected and centrifuged at 10000 g for 20 min.

5. The supernatant was collected and filtered at 0.45 um, to obtain a protein supernatant of wild-type hNGF and its mutants.

6. SDS-PAGE detection, quantification by silver nitrate staining.

### Example 4: Purification of Wild-Type hNGF and its Mutants

The protein supernatant of the NGF and its mutants obtained in the above Example 3 was purified.

1. Cation exchange chromatography: the protein supernatant of wild-type hNGF and its mutants was first adjusted to pH 4.0 with acetic acid and water. A CM Sepharose FF column was fully equilibrated with 0.05 mol/L acetate buffer (pH 4.0) and then loaded. After the loading was completed, it was rinsed with an equilibration solution to the baseline, and then impure peaks were eluted to the baseline with an equilibration solution of 0.05 mol/L Tris-HCl (pH 9.0), and finally subjected to a gradient elution with 0.05 mol/L Tris-HCl and 0.05 mol/L Tris-HCl-0.4 mol/L NaCl (pH 9.0). The target peak was collected according to the ultraviolet absorption, in which the collection was started when the number shown on the UV detector began to rise, and stopped when the number was lowered to the baseline.

2. Hydrophobic chromatography: a Butyl Sepharose 4 FF column was well equilibrated with 0.02 mol/L phosphate (pH 6.8)-1.5 mol/L sodium chloride buffer. Into the target peak solution collected in step 1, a sodium chloride solid was added, such that the final concentration of sodium chloride in the solution was 1.5 mol/L. After the sodium chloride was fully dissolved, the sample was loaded at a speed of 120 cm/h. After the loading was completed, it was rinsed with an equilibration solution to the baseline, and then the target peak was collected by elution with 0.02 mol/L phosphate (pH 6.8).

3. Gel exclusion chromatography: Superdex 75 prep grade chromatography column was fully equilibrated with 0.05 mol/L phosphate-0.15 mol/L sodium chloride buffer at pH 6.8. Then, the target peak collected in step 2 was loaded, in which the collection was started when the number shown on the UV detector began to rise from the baseline, and stopped when the number was lowered to the baseline.

The SDS PAGE of the wild-type hNGF purified by Superdex 75 column is shown in FIG. 1, indicating that the prepared NGF has high purity. The samples with target protein peaks of the collected wild-type hNGF and its mutants were concentrated to 0.4 mg/ml by using an ultrafiltration tube, and stored at 4° C. for subsequent experiments.

### Example 5: Measurement for the Activity of Wild-Type hNGF and its Mutants by Chicken Embryo Method

1. Measurement for the Activity of Wild-Type hNGF and its Mutants Activity by Chicken Embryo Dorsal Root Ganglion Method

The wild-type hNGF (amino acid sequence is shown in the sequence listing) and its mutants (amino acid sequences were shown in the sequence listing) samples obtained in the above Example 4 were diluted. A solution: 6 ng of the extracted wild-type hNGF and its mutants samples were dissolved by adding 1 ml of a serum-free DMEM medium. B solution: 50 μl of A solution was added with 4.95 ml of serum-free DMEM medium. C solution: 60 μl of B solution was added with 2.94 ml of serum-free DMEM (3 ml in total) to achieve a final concentration (3 AU/ml). A and B solutions were diluted in a centrifuge tube, and C solution was placed in a cell bottle. C solution was used as a No. 1 bottle, and was further diluted by a factor of 3 to No. 2, No. 3, No. 4, No. 5 and No. 6 solutions to be tested. Each solution to be tested was added into one culture bottle at 2 ml/bottle. At the same time, a serum-free DMEM culture medium was used as a blank control, and the standard product purchased from the National Institute of Food and Drug Control was used as a positive control (reference product). After an 8-day-old chicken embryo dorsal root ganglion was added, the culture bottle was placed in a saturated humidity incubator in a 5% CO₂ and at 37° C., and the results were observed after 24 h.

The content of NGF per ml of the sample to be tested when growing best is used as 1 activity unit (AU). The titer was calculated from end-point judgment, which was deemed as the best dilution for growing taken from the 3rd and 4th dilutions back counted from the dilution having the negative control result. The reference product is a standard product purchased from the National Institute of Food and Drug Control, in which the capacity of each is 1000 AU.

The formula for calculating the specific activity of NGF is shown as follow:

specific activity of the sample to be tested (AU/mg)=activity of the reference product (AU/ml)×[pre-dilution factor of the sampler×activity at the dilution point of the corresponding reference product (AU/ml)/actual activity of the reference product (AU/ml)]

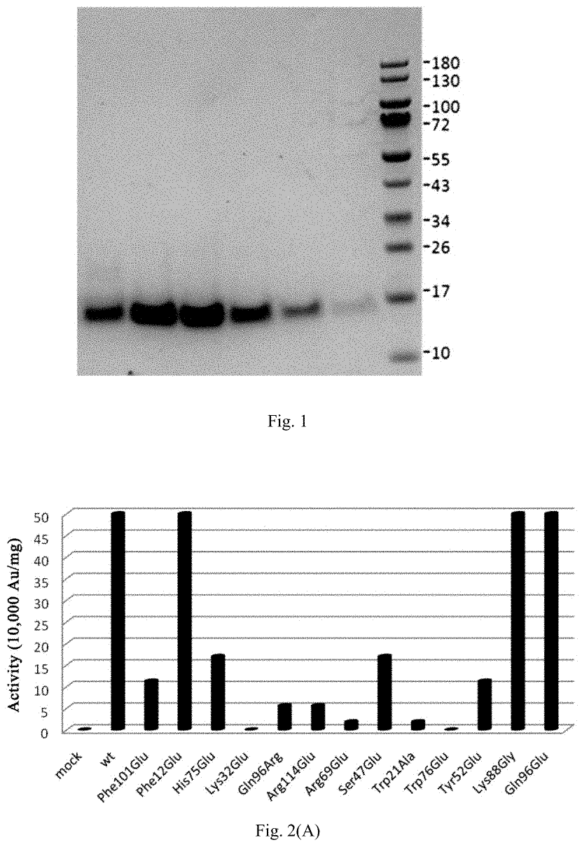

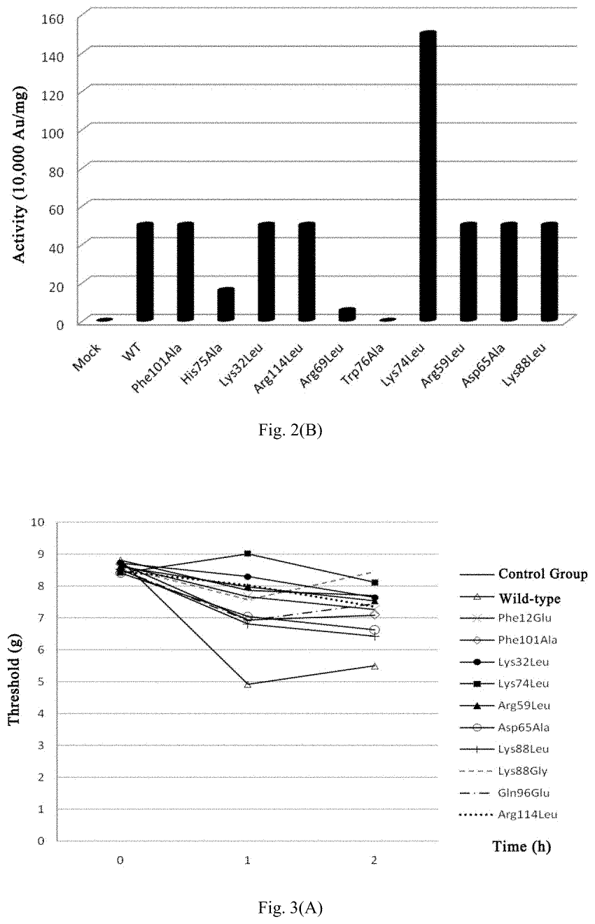

The results of the measurements are shown in FIGS. 2(A) and 2(B). The results showed that the hNGF mutants Phe12Glu, Lys32Leu, Arg59Leu, Asp65Ala, Lys74Leu, Lys88Leu, Lys88Gly, Gln96Glu, Phe101Ala, Arg114Leu all retained wild-type activity and even a higher activity.

### Example 6: Measurement of the Activity of NGF and its Mutants by TF-1 Cell Method

The detailed operation method was performed in accordance with the method in Example 1 of a patent entitled “Method for Quantitatively Measuring Nerve Growth Factor Activity” with a publication number of CN103376248A, and the test results of the specific activity were shown in the following table.

TABLE 1

| | Specific Activity (U/mg) |
|---|---|
| Sample Name | by Cell Method |
| Wild-type hNGF | 430,000 |
| Lys74Leu | 767,000 |
| Phe12Glu | 620,000 |
| Lys88Gly | 590,000 |
| Gln96Glu | 430,000 |

### Example 7: Detection for Whether NGF and its Mutants Cause Pain (Pain Threshold)

Experimental principle: qualified mouse having a normal response to pain was screened, and injected a certain dose of NGF sample (wild-type or its mutants). The pain threshold of curved claw response in mouse by mechanical stimulation was determined, and subjected to a statistical analysis, and finally whether the sample caused mouse hyperalgesia was determined.

7-1. Observation of Short-Term Pain-Causing Condition

I. Experimental Material

Dynamic Plantar Aesthesiometer (Ugo Basile, Italy), model 37450.

II. Experiments

1. Screening of Qualified Mice

SPF grade CD-1 mice were ordered, in which the mice were male weighed 30-35 g.

By the Dynamic Plantar Aesthesiometer [Ugo Basile, Italy, model: 37450], the experimental animals were screened for qualified mice, in which the mean threshold of the left and right feet is between 7.5 and 10 and the P value for the threshold of the left and right feet in a same mouse is more than 0.05.

Mice were randomly divided into experimental groups and blank control groups, in which the experimental groups were divided into subgroups according to various samples and administration doses, and each group had 10 mice.

2. Design of Administration for NGF Samples

2.1. Screening of the Pain-Causing Dosage of Wild-Type Samples

Drug formulation: a positive control NGF wild-type samples and each mutant sample were diluted by using sample stock solutions (50 mM PB, 150 mM NaCl, pH 6.8).

Blank control: stock solution of NGF samples.

Mode of administration and dosage: 20 μl were administered plantar subcutaneously to the left and right feet of mice respectively.

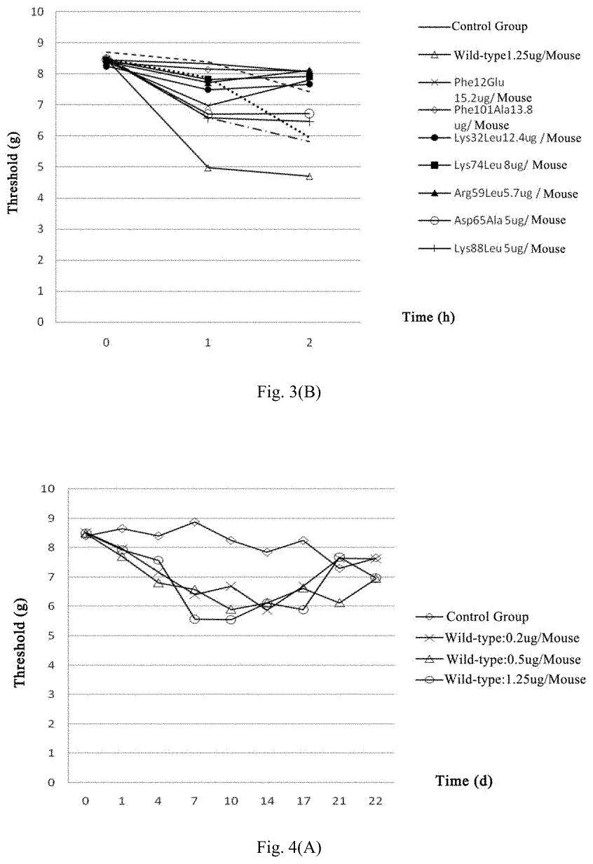

The minimum dose for a short-term administration was 1.25 μg per mouse, while the corresponding higher dose was administered to determine the ability to cause pain, see the dose labeled in FIG. 3 (B).

3. Measurement of Pain Threshold

Mechanical threshold measurements were performed at 1 h and 2 h after administration respectively, and values were recorded for observing the short-term administration (within 2 h).

4. Statistical Analysis for the Results

GraphPad Prism software was used for graph drawing and statistical analysis for the results. The difference in the mechanical thresholds between each dose group and the control group were compared, and the ability to cause pain of wild-type samples and mutants samples were analyzed.

As can be seen from FIG. 3(A), when the minimum dose of administration was 1.25 μg per mouse, the control group had no pain, while the wild-type positive control group had a pain threshold of obviously less than 5 at 1 h, and the pain was obvious; the pain threshold for each mutant experimental group was about 7, and there was no significant difference as compared with the negative control group, indicating that the short-term injection of mutants were basically no painful;

As can be seen from FIG. 3(B), when the administration dose of the control group and the wild-type was 1.25 μg per mouse and the administration dose of the experimental group was increased, the control group had no pain, while the wild-type positive control group had a pain threshold of obviously less than 5 at 1 h, and the pain was obvious; the pain threshold for each mutant experimental group was about 7, and there was no significant difference as compared with the negative control group, indicating that the short-term injection of mutants were also basically no painful in the case of increasing the administration dose.

7-2. Observation of Long-Term Pain-Causing Condition

Three of the above mutants No. 1 (Phe12Glu), No. 2 (Lys88Gly) and No. 3 (Arg114Leu) were randomly selected for long-term pain-causing test. Except that the three doses for this experiment were: 0.2 μg per mouse, 0.5 μg per mouse and 1.25 μg per mouse, once a day for 3 weeks, during which the pain threshold was measured continuously, the rest all were performed according to the method in 7-1 “observation of short-term pain-causing condition”.

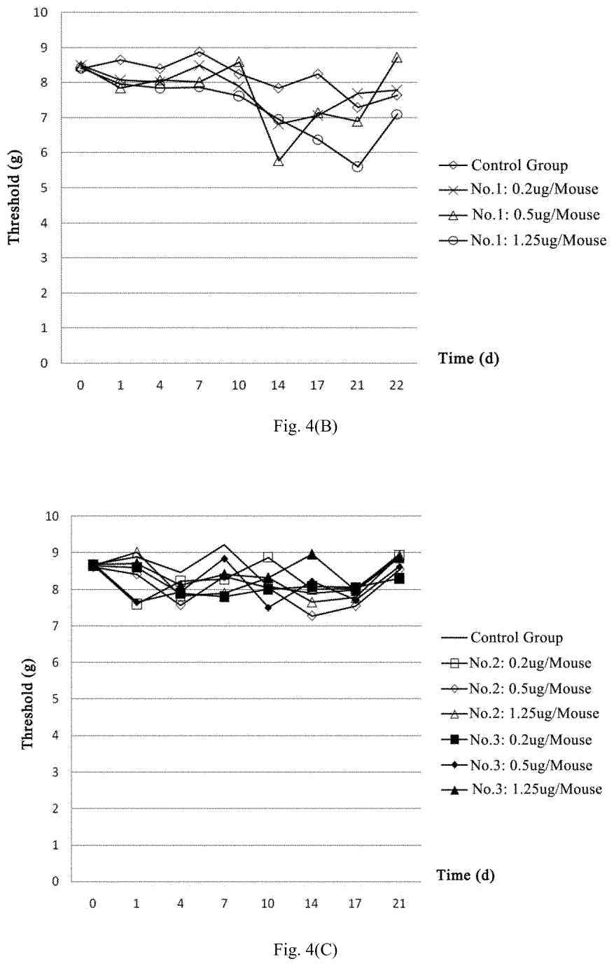

The results are shown in FIGS. 4(A), 4(B) and 4(C), indicating that as for the mutant Phe12Glu, the pain threshold was not significantly reduced within 14 days, and no pain was observed, while only a short-term pain was observed for a medium dose (0.5 μg per mouse) after 14 days; as for the mutant Lys88Gly and the mutant Arg114Leu, no obvious pain threshold change was showed during the test; and as for the wild-type NGF, the pain thresholds of three doses were reduced within 17 days gradually, and pain gradually appeared. In view of this, the mutants of the present disclosure have a significant pain reducing effect over the wild-type.

### Example 8. Behavioral Test of Whether Wild-Type hNGF and its Mutants Cause Pain

The wild-type hNGF and its mutants samples were administered in the joints of the mice, and whether the samples cause pain were examined by leg lifting maintenance time and number of the mice according to behavior.

Experiments

1. Ordering of Mouse

SPF grade CD-1 mice were ordered, in which the mice were male weighed 30-35 g. They were randomly divided into experimental groups, blank control groups (abbreviated as control groups) and positive control groups, in which each group was divided into seven subgroups according to the dose, and each group was randomly selected for 6 mice.

2. Administration Dose and Time

2.1. Administration Dose

Positive control: the wild-type hNGF was diluted with a sample stock solution (50 mM PB, 150 mM NaCl, pH 6.8) to 1.25 μg/10 μl group;

Experimental group: the preparation method of the mutant drug was the same as the positive control, and the mutants Phe12Glu, Lys88Gly and Arg114Leu were diluted to 1.25 μg/10 μl group and 0.5 μg/10 μl group;

Blank Control: Normal Saline.

2.2. Mode of Administration

Drugs were injected into the joint in hind legs of the mice, in which 10 μl was administered into each joint cavity.

2.3. Time of Administration

Each dose group was administered in a single continuous 3-4 days. That is, the administration was performed at 10 am on the first day, and at the same time points on the 2nd, 3rd, and 4th days thereafter.

3. Behavioral Observation

Observation was performed at 2nd and 4th hours after the administration of each experimental group, and at the same time points on the 2nd, 3rd, and 4th days of administration.

Observation indicators: the numbers of the mouse spontaneous leg lifting within 2 min and the maintenance time (s) of each leg lifting were used to calculate the accumulated time of leg lifting.

4. Statistical Analysis for the Results

A two-way ANOVA of GraphPad Prism software was used to compare the leg lifting maintenance time, and analyze whether different samples cause pain.

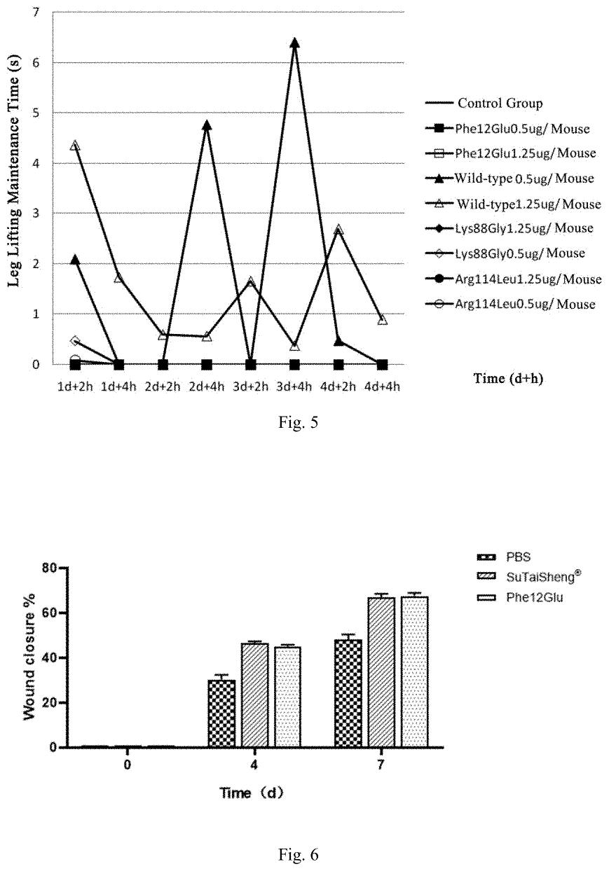

The experimental results are shown in FIG. 5. The wild-type hNGF group may cause obvious pain at each time point after the injection of the drug. There is no leg-lifting behavior or pain abnormality in the continuous administration of each dose group of the mutant, thereby determining that the mutants did not cause pain. Chi-square analysis showed that there was a significant difference between the positive control group and the experimental group at each detection time point.

### Example 9: NGF Promotes Wound Healing of Diabetic Neuropathy

Diabetic neuropathy is one of the common chronic complications of diabetes, the patients of which are characterized by slow wound healing, different degrees of infection, ulcers, and anthrax, even with a risk of amputation. This Example illustrates the study of therapeutic effects of NGF in a diabetic neuropathy animal model (e.g., through evaluation of wound healing).

CD-1 mice were obtained from Beijing Vital River Laboratory Animal Technology Co., Ltd. Animal model of diabetes was established with standard methods (e.g., see G. Graiani et al., “Nerve growth factor promotes reparative angiogenesis and inhibits endothelial apoptosis in cutaneous wounds of Type 1 diabetic mice.” Diabetologia. 2004, 47(6):1047-54). After 4 weeks of induction of diabetes, mice were anesthetized, and two full-thickness skin wounds of 4 mm in diameter were generated side by side in interscapular region by a disposable skin punch equipment. 50 μg/mL SuTaiSheng® mouse NGF (Staidson (Beijing) Biopharmaceuticals Co., Ltd.) or Phe12Glu was administered to the right wound with a dose of 20 μL/administration. PBS of equal volume was administered to the left wound (serving as negative control). PBS, SuTaiSheng® mouse NGF, or Phe12Glu was administered on Days 0 (after drilling mice on the back), 1, 2, and 3, once per day. The wound area was measured immediately after drilling and recorded as wound area of Day 0. Then wound areas on Days 4 and 7 were measured followed by calculating wound closure rate. Recorded data were analyzed by Student t test. Histogram was drawn by GraphPad Prism 8.0.1.

As shown in FIG. 6, would closure on Day 7 was improved compared to Day 4 for all groups; SuTaiSheng® mouse NGF and Phe12Glu all promoted diabetic wound healing significantly, when compared to PBS negative control (p<0.01). Specifically, on Day 4, the average wound area in PBS treatment group was about 1.3 times bigger than that in SuTaiSheng® mouse NGF or Phe12Glu treatment group. These results demonstrate that SuTaiSheng® mouse NGF or Phe12Glu can effectively improve the slow wound healing defect in diabetic mice.

### Example 10: Therapeutic Effect of NGF on Alzheimer's Disease

Alzheimer's disease (AD) is a degenerative disease of central nervous system with progressive memory loss as main clinical manifestation, which mostly occurs in the elderly with complex pathogenesis. This Example illustrates the study of therapeutic effects of NGF (SuTaiSheng® mouse NGF and Phe12Glu) on AD in vivo (e.g., through evaluation of animal behavioral changes).

Wistar rats were used to establish animal model of AD with standard methods (for example, see G. L. Wenk et al. “Basal forebrain neurons and memory: a biochemical, histological and behavioural study of differential vulnerability to ibotenate and quisqualate.” Behav Neurosci, 1992, 106(6): 909-923). Briefly, Wistar rats received stereotactic injection of ibotenic acid (IBO). Two days after IBO administration, anesthetized AD model rats were placed on their backs. 150 μg/mL NGF (SuTaiSheng® mouse NGF or Phe12Glu) was administrated intranasally at a total dose of 100 μL/administration. AD model rats administrated with PBS of equal volume served as negative control group. NGF or PBS was administrated once per day for 7 days continuously. The behavioral changes of rats were evaluated by Morris water maze (MWM) on Day 7. Briefly, using a Morris water maze device, rats were trained to climb the platform before experiment. The platform seeking time (escape latency; from entering the water to climbing onto the platform), and the times of crossing the position where the platform had been placed but then removed within 120s, were recorded during the experiment. Recorded data were analyzed by Student t test.

As shown in Table 2, the platform seeking time of AD model rats treated by SuTaiSheng® mouse NGF or Phe12Glu was significantly shortened (*p<0.05), and the platform crossing times were significantly increased (*p<0.05), compared to the negative control group. Thus, SuTaiSheng® mouse NGF and Phe12Glu described herein can both effectively improve spatial cognition, memory, and learning ability of AD model rats.

TABLE 2
Statistical table of Morris water maze (x̅ ± s)

| | Platform seeking | Platform crossing |
|---|---|---|
| Group | time (s) | times(times) |
| Negative control | 52.28 ± 20.12 | 3.31 ± 1.85 |
| SuTaiSheng ® mouse NGF | 32.05 ± 16.36* | 7.43 ± 2.85* |
| Phe12Glu | 30.97 ± 15.26* | 7.12 ± 2.86* |

### Example 11: Therapeutic Effect of NGF on Neurotrophic Keratitis

Neurotrophic keratitis is a degenerative disease caused by corneal epithelial healing disorder, which is mainly characterized by decreased corneal sensitivity. This Example illustrates the study of the therapeutic effects of NGF (SuTaiSheng® mouse NGF or Phe12Glu) on neurotrophic keratitis in a neurotrophic keratitis rat model (e.g., by corneal fluorescein sodium staining assay or corneal nerve length measurement).

To establish the neurotrophic keratitis animal model, 3-day-old SD rats were subcutaneously injected with 8 mg/ml capsaicin solution (Shanghai McLean Biochemical Technology Co., Ltd., #C10831884) at a dose of 50 μl per rat. Two weeks after capsaicin injection, 60 μg/ml NGF (SuTaiSheng® mouse NGF or Phe12Glu) was administered 6 times a day with an interval of about 2 hours in the form of eye drops, 20 μl/eye/administration. Equal volume of 0.9% sodium chloride solution was administered at the same frequency as negative control. The first day of administration was denoted as D1. The treatment lasted for 2 weeks, and was performed only once in each group on D15.

Corneal fluorescein sodium staining was then carried out to evaluate the therapeutic effects of NGF. Corneal fluorescein sodium staining score can directly indicate the integrity and damage degree of cornea. Intact cornea cannot be stained. Only damaged cornea can be stained, and the higher the staining score, the higher the degree of corneal damage. Briefly, the fluorescein sodium solution (3 μL, 0.5%) was dropped into rats' eyes, and the eyes were stained for 1.5 min. Then the conjunctival sac was rinsed with 1.25 mL sterile normal saline every 10 seconds for 3 consecutive times. After each wash, the normal saline around eyes was sopped up with paper tissue. 5 minutes after staining, the ocular surface was observed in slit lamp (with cobalt blue filter), and photos were taken and scored. An improved NEI scale for grading fluorescent staining was used as the scoring standard. Specifically, each cornea was divided into 5 areas (1-central area, 2-upper, 3-temporal, 4-nasal, and 5-lower), the top score for each area was 8 points, wherein point 0 indicated no colored area, 1 indicated the spot colored area was 1%˜25% of the corresponding area, 2 indicated the spot colored area was 26%˜50% of the corresponding area, 3 indicated the spot colored area was 51%˜75% of the corresponding area, and 4 indicated the spot colored area was 76%˜100% of the corresponding area. If the colored area was dense and/or obvious fusion in the area can be seen, 1, 2, 3, or 4 points would be further given according to the percentage of the colored area in the corresponding area, respectively, i.e. 1 extra point for colored area of 1%˜25%, 2 extra points for colored area of 26%˜50%, 3 extra points for colored area of 51%˜75%, and 4 extra points for colored area of 76%˜100%. The maximum total score of each eye was 40 points. The measurements were taken 4 times in total on Days 0 (before the first treatment), 4, 8, and 14. Total score of corneal fluorescein sodium staining was calculated. Recorded data were analyzed by SPSS13.0, and histogram was drawn using GraphPad Prism 8.0.1.

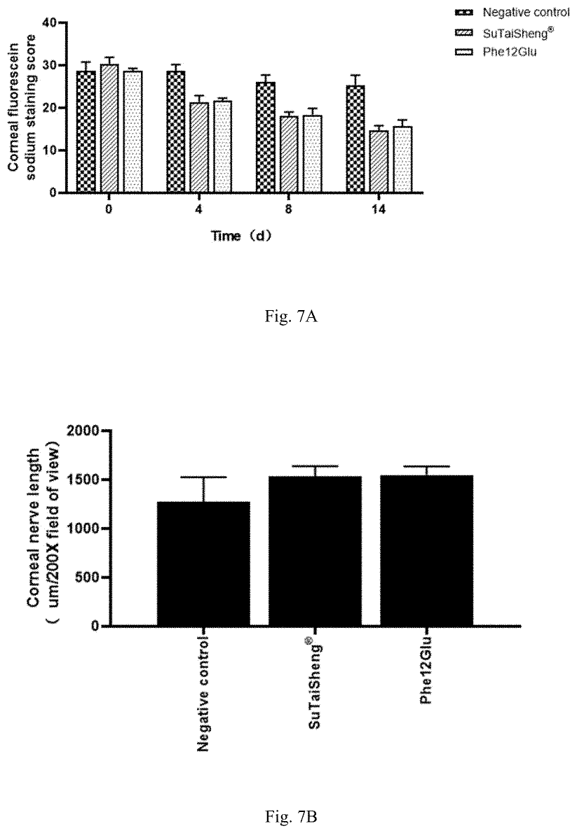

As shown in FIG. 7A, the corneal fluorescein sodium staining scores of neurotrophic keratitis model rats in NGF (SuTaiSheng® mouse NGF or Phe12Glu) treated experimental group were significantly lower (p<0.01 on Day 4 and 8, p<0.001 on Day 14) than that in the negative control group, indicating that NGF (SuTaiSheng® mouse NGF or Phe12Glu) can significantly restore the integrity of damaged cornea.

A corneal nerve counting assay was further carried out to study the therapeutic efficacy. On D15, rats were sacrificed 1 hour after the last treatment. The right eyeball was harvested. Cornea was separated along the corneal limbus, washed, paved, stained, and fixed on glass slides. The morphology of corneal nerve fibers was observed under optical microscope (200×). The area of cornea was divided radially from the center into four flaps, followed by taking photos of a vision field with the most corneal nerves in each of the four flaps. Then, the length of corneal nerves in each vision field was measured, and the average length of corneal nerves in all four fields was calculated as the final result. SPSS13.0 was used to process the data, and graphpad prism 8.0.1 was used to draw the histogram.

As shown in FIG. 7B, the average corneal nerve length of rat models of neurotrophic keratitis treated with NGF (SuTaiSheng® mouse NGF or Phe12Glu) was significantly longer (p<0.05) than that in the negative control group. Specifically, the average corneal nerve length of rats treated by SuTaiSheng® mouse NGF or Phe12Glu was about 1.14 times, 1.14 times of that in negative control group, respectively. These results demonstrate that NGF (SuTaiSheng® mouse NGF or Phe12Glu) can effectively remedy the damage of corneal nerve caused by neurotrophic keratitis.

What is claimed is:

1. A method of using a nerve growth factor mutant to treat or inhibit a nervous system disease comprising:
administering the nerve growth factor mutant to a subject in need thereof,
wherein the nerve growth factor mutant comprising Phe12Glu with reference to the amino acid positions set forth in a wild-type human nerve growth factor.

2. The method of claim 1, wherein the nervous system disease is a disease associated with neuronal degeneration or injury in the central and/or peripheral nervous system.

3. The method of claim 1, wherein the wild-type human nerve growth factor comprises an amino acid sequence of SEQ ID No: 2.

4. The method of claim 1, wherein the nervous system disease is selected from the group consisting of: diabetic neuropathy, Alzheimer's disease, and neurotrophic keratitis.

5. The method of claim 1, wherein using the nerve growth factor mutant to improve a subject in one or more neurological diseases: diabetic neuropathy, Alzheimer's disease or neurotrophic keratitis.

6. The method of claim 5, wherein using the nerve growth factor mutant to improve wound healing in diabeties, improve the spatial cognition and the ability of learning and memory of Alzheimer's subject, restore the integrity of subject's cornea, or improve the damage of corneal nerve caused by neurotrophic keratitis.

7. A use of a nerve growth factor mutant in the preparation of a medicament for treating or inhibiting a nervous system disease:
wherein the nerve growth factor mutant comprising Phe12Glu with reference to the amino acid positions set forth in a wild-type human nerve growth factor.

8. The use of claim 7, wherein the nervous system disease is a disease associated with neuronal degeneration or injury in the central and/or peripheral nervous system.

9. The use of claim 7, wherein the wild-type human nerve growth factor comprises an amino acid sequence of SEQ ID No: 2.

10. The use of claim 7, wherein the nervous system disease is selected from the group consisting of: diabetic neuropathy, Alzheimer's disease, and neurotrophic keratitis.

11. The use of claim 7, wherein the medicament improves a subject in one or more neurological diseases: diabetic neuropathy, Alzheimer's disease or neurotrophic keratitis.

12. The use of claim 11, wherein the medicament improves wound healing in diabeties, improves the spatial cognition and the ability of learning and memory of Alzheimer's subject, restores the integrity of subject's cornea, or improves the damage of corneal nerve caused by neurotrophic keratitis.
